(19) United States
(12) Patent Application Publication
(10) Pub. No.: US 2025/0232172 A1
(43) Pub. Date: Jul. 17, 2025

(54) METHOD AND APPARATUS FOR EMPLOYING GRAPH NEURAL NETWORK BY USING PLURALITY OF PROCESSORS

(71) Applicants: SAMSUNG ELECTRONICS CO., LTD., SUWON-SI (KR); Seoul National University R&DB Foundation, Seoul (KR)

(72) Inventors: SAEROM CHOI, SUWON-SI (KR); Yutack Park, SEOUL (KR); Jaesun Kim, SEOUL (KR); Seungwu Han, SEOUL (KR)

(21) Appl. No.: 18/809,503

(22) Filed: Aug. 20, 2024

(30) Foreign Application Priority Data
Jan. 12, 2024 (KR) 10-2024-0005283

(51) Int. Cl. G06N 3/08; G06N 3/042

(52) U.S. Cl. CPC G06N 3/08; G06N 3/042

(57) ABSTRACT

A method including obtaining an input graph including a plurality of network components, where each of the plurality of network components include a plurality of nodes and a plurality of edges, segmenting the input graph into a plurality of partial input graphs, where the plurality of partial input graphs includes a first partial input graph and a second partial input graph, generating network features based on the plurality of partial input graphs, where each of the network features includes a connectivity relation between a network component and an adjacent network component among the plurality of network components in the input graph, transmitting a network feature from the first partial input graph to an adjacent network feature in the second partial input graph to obtain an aggregated network feature, and updating the network features of the plurality of network components based on the aggregated network feature.

### CROSS-REFERENCE TO RELATED APPLICATION

This U.S. non-provisional patent application claims priority under 35 U.S.C. § 119 to Korean Patent Application No. 10-2024-0005283, filed on Jan. 12, 2024, in the Korean Intellectual Property Office, the disclosure of which is incorporated by reference herein in its entirety.

### BACKGROUND

One or more embodiments of the disclosure relate to processing the application of a machine-learning model using a plurality of processors.

Network data (or graph data) refers to useful data for describing various objects and a relationship among the objects. For example, network data includes a social network, a relational network, a molecular structure, or a recommender system. The network data may include nodes respectively corresponding to the objects and an edge connecting the nodes according to a relationship among the nodes such that a correlation among the objects may be analyzed. For example, a node may represent a user and an edge may represent a relationship between users in a social network (e.g., friends). For example, a node may represent an individual paper and an edge may represent a citation relationship in a relational network. In some cases, a node may represent a user or a product and an edge may represent a recommendation relationship in a recommender system.

A subfield in network data is network embedding technology using an artificial neural network, such as a graph neural network (GNN). Network embedding technology has been developed to express a similarity among nodes by vectorizing each of the nodes in network data. When using a network embedding method, each of the nodes is vectorized and placed in a low-dimensional embedding space, and the similarity among the nodes may be recognized based on a distance between the nodes placed in the embedding space. In some cases, the network embedding method is used for network data. For example, network embedding method enables a new state of each node to be easily updated by exchanging information through an edge connected among the nodes, combining the information of the nodes with the phase information of a network, and expressing the combined information in the embedding space.

### SUMMARY

A method including obtaining an input graph including a plurality of network components, where each of the plurality of network components include a plurality of nodes and a plurality of edges; segmenting the input graph into a plurality of partial input graphs, wherein the plurality of partial input graphs includes a first partial input graph and a second partial input graph; generating, using a plurality of processors and a first layer of a graph neural network (GNN), network features based on the plurality of partial input graphs, wherein each of the network features includes a connectivity relation between a network component and an adjacent network component among the plurality of network components in the input graph; transmitting, among the plurality of processors, a network feature from the first partial input graph to an adjacent network feature in the second partial input graph to obtain an aggregated network feature; and updating, using the plurality of processors and a second layer of the GNN, the network features of the plurality of network components based on the aggregated network feature.

A method including obtaining an input graph including a plurality of network components, where each of the plurality of network components include a plurality of nodes and a plurality of edges; segmenting the input graph into a first partial input graph and a second partial input graph; generating, using a first processor and a graph neural network (GNN), first network features based on the first partial input graph, wherein each of the first network features includes a connectivity relation between a first network component and an adjacent first network component among the plurality of network components in the input graph; generating, using a second processor and the GNN, second network features based on the second partial input graph, wherein each of the second network features includes a connectivity relation between a second network component and an adjacent second network component among the plurality of network components in the input graph; transmitting, among the first processor and the second processor, a network feature from the first partial input graph to an adjacent network feature in the second partial input graph to obtain an aggregated network feature; and updating, using the second processor and a second layer of the GNN, the second network features based on the aggregated network feature.

An electronic device including a plurality of processors, wherein the plurality of processors is configured to: obtain an input graph including a plurality of network components, where each of the plurality of network components include a plurality of nodes and a plurality of edges as a plurality of components, segment the input graph into a plurality of partial input graphs, wherein the plurality of partial input graphs includes a first partial input graph and a second partial input graph, generate network features based on the plurality of partial input graphs, wherein each of the network features includes a connectivity relation between a network component and an adjacent network component among the plurality of network components in the input graph, transmit, among the plurality of processors, a network feature from the first partial input graph to an adjacent network feature in the second partial input graph to obtain an aggregated network feature, and update the network feature of the plurality of network components based on the aggregated network feature.

### BRIEF DESCRIPTION OF THE DRAWINGS

FIG. 1 illustrates an example of an input graph and a graph neural network (GNN) according to embodiments of the present disclosure.

FIG. 2 illustrates an example of the input graph and the GNN according to embodiments of the present disclosure.

FIG. 3 illustrates an example of a method for applying an input graph to a GNN using an electronic device according to embodiments of the present disclosure.

FIG. 4 illustrates an example of an operation of the electronic device segmenting an input graph into a plurality of partial input graphs according to embodiments of the present disclosure.

FIG. 5A illustrates an example of an operation of the electronic device applying a plurality of partial input graphs, in parallel state, to a GNN according to embodiments of the present disclosure.

FIG. 5B illustrates an example of an operation of the electronic device applying a plurality of partial input graphs, in parallel state, to a GNN according to embodiments of the present disclosure.

FIG. 6 illustrates an example of a method for a plurality of processors of the electronic device employing GNNs having different operation amounts according to embodiments of the present disclosure.

FIGS. 7A and 7B illustrate examples of an operation of the electronic device performing forward propagation and backward propagation on a GNN according to embodiments of the present disclosure.

FIG. 8 illustrates an example of a configuration of the electronic device according to embodiments of the present disclosure.

FIG. 9 illustrates an example of a method for processing graphical data using an electronic device according to embodiments of the present disclosure.

### DETAILED DESCRIPTION

The following detailed structural or functional description is provided as an example and various alterations and/or modifications may be made to the embodiments. Accordingly, the embodiments are not construed as limited to the disclosure and should be understood to include all changes, equivalents, and replacements within the inventive concept and the technical scope of the disclosure.

Terms, such as first, second, and the like, may be used herein to describe various components. Each of these terminologies is not used to define an essence, order or sequence of a corresponding component but used merely to distinguish the corresponding component from other component(s). For example, a first component may be referred to as a second component, and similarly the second component may also be referred to as the first component.

In some cases, when a first component is “connected”, “coupled”, or “joined” to a second component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. It may be further understood that the terms “comprises/including” and/or “includes/including” used herein specify the presence of stated features, integers, steps, operations, elements, and/or components, but do not preclude the presence or addition of one or more other features, integers, steps, operations, elements, components and/or groups thereof. In some cases, the term “a plurality” may refer to one or more. For example, the phrase a plurality of elements may refer to one or more elements.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art and are not to be interpreted in an idealized or overly formal sense unless expressly so defined herein.

Hereinafter, embodiments of the present disclosure are described in detail with reference to the accompanying drawings. When describing the embodiments with reference to the accompanying drawings, like reference numerals refer to like elements and a repeated description related thereto may be omitted. The drawings might not be to scale, and the relative size, proportions, and depiction of elements in the drawings may be exaggerated for clarity, illustration, and convenience.

A graph neural network (GNN) is a type of neural network designed to operate on graph-structured data. In some cases, the GNN can process various sizes of graphs having different levels of complexities. For example, the nodes of the GNN represent entities depicted in the graph-structured data, and the edges of the GNN represent relationships between the entities depicted in the graph-structured data. In some aspects, a GNN uses the graph structure to aggregate and propagate information across nodes, and captures local and global patterns within the graph-structured data.

GNNs are used in various applications such as social network analysis, recommendation systems, biological and chemical networks, and transportation networks. For example, in social network analysis, a GNN is used to identify communities and predict user behaviors. For example, in recommendation systems, a GNN is used to suggest items based on user-item interaction graphs. For example, in biological and chemical networks, a GNN is used to model molecular structures and predict properties of compounds. For example, in transportation networks, a GNN is used for traffic prediction and route optimizations. However, the processing time for processing graph-structured data using a GNN can be lengthy based on the data size. In some cases, a processor may be unable to handle the data due to the massive size of the graph-structured data.

In some cases, a processor is used to process graph-structured data in a GNN. In some cases, the processor can perform one task at a time, which decreases the processing speeds. Thus, the wait time may be extended as the processor sequentially processes the graph-structured data. In some cases, a single processor might not be able to handle complex computations or parallel tasks, which may further increase the latency. In some cases, as the single processor processes a large volume of graph-structured data, system failure or potential crashes may appear. In some cases, two or more processors may be used to process graph-structured data in parallel processing. However, conventional methods of parallel processing may fall short in handling large simulations such as molecular dynamics.

Embodiments of the present disclosure provides a method and an electronic device that improve on conventional models by efficiently generate a predicted output data using a plurality of processors and a GNN. For example, by segmenting the input graphical data into a plurality of partial input graphs and using a plurality of processor to generate network features based on the plurality of partial input graphs, the processing speed of the system can be reduced. In some cases, the electronic device generates first network features of the first node group of a first partial input graph and generates second network features of the second node group of a second partial input graph in a parallel state. By simultaneously computing the first network features and the second network features using the first processor and the second processor, respectively, the processing speed can be reduced. In some embodiments, the electronic device determines a first network component group and a second network component group based on the characteristic (e.g., an operation amount) of the processor. By generating the first partial input graph and the second partial input graph based on the first network component group and a second network component group, respectively, the electronic device is able to optimize the computational resources to generate an output data (e.g., a prediction) using the GNN.

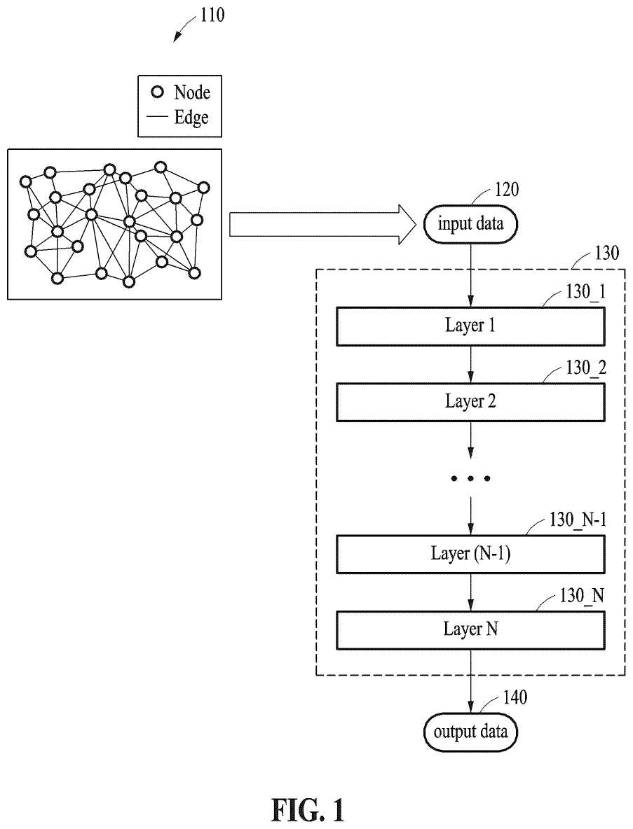

FIG. 1 illustrates an example of an input graph and a graph neural network (GNN) according to embodiments of the present disclosure. The example, shown includes input graph 110, input data 120, GNN 130, and output data 140. In one aspect, GNN 130 includes a plurality of layers.

An input graph 110 may include information about a plurality of entities included in a target and relations among the plurality of entities. The input graph 110 may include a plurality of nodes (represented in circles) and a plurality of edges (represented in lines). Hereinafter, a component of the input graph 110 may be used as a term including a node of the input graph 110 and an edge of the input graph 110. In some cases, for example, the node may correspond to an entity included in the target and the edge may correspond to a relation between entities (or two or more nodes).

According to an embodiment, the target may include a matter, an entity of the target may be an element (e.g., an atom, a molecule, an electron, a proton, or a neutron) that forms the matter. In some cases, a relation between elements may be a distance between the elements and/or a bond between the elements.

However, in some cases, the target is not limited to a matter and may include living things (e.g., people, animals, or plants) and inanimate objects (e.g., automobiles, motorcycles, or bicycles) that may move, change in movement, and/or change in shape.

A GNN 130 may be a type of a machine learning model and may be a model generated or trained to generate output data 140. For example, the output data 140 indicates information from the input graph 110 included in input data 120. For example, the input data 120 corresponds to the input graph 110 including the nodes and the edges. The input data 120 may include information of each node and each edge of the input graph 110 (sometimes referred to as the graph 110). The input data 120 may further include a connectivity relation among components with the information on each component (e.g., each node or each edge) of the graph 110. In some embodiments of the present disclosure, the connectivity relation among the components of the graph 110 may also be expressed by a connectivity relation of the graph 110. The output data 140 may include a feature of each node and a feature of each edge of the graph 110.

In some aspects, the GNN 130 may include one or more of a graph convolutional network (GCN), a graph attention network, message passing, a graph isomorphism network (GIN), or a directed message passing neural network (D-MPNN), but examples are not limited to the foregoing examples.

GCN is a type of neural network architecture that handles graph-structured data, where data points are represented as nodes connected by edges. GCNs operate by iteratively aggregating and transforming features from a node's local neighborhood, and enables nodes to learn from the corresponding features and the structure of the graph. In one aspect, a convolution operation is used to generalize the concept of convolution from grid-based data to graphs.

Graph attention network is a type of neural network designed for graph-structured data. In some cases, graph attention network includes an attention mechanism. For example, each node in the graph learns to focus on the most relevant parts of the neighborhood during the feature aggregation process. This process can be performed using self-attention, where attention coefficients are computed for each pair of connected nodes. The attention coefficient indicates the importance of one node's features to another. Then, the attention coefficients are used to weigh the neighboring features before the features are aggregated. This attention mechanism enables the model to dynamically assign different levels of importance to different neighbors. Thus, the model is able to capture complex relationships and heterogeneity in the graph.

In GNNs, message passing refers to information that is iteratively exchanged between nodes to learn effective representations of the graph's structure and node features. In the message passing framework, each node in the graph updates representation of each node by aggregating information (messages) from the neighboring nodes. In some cases, the message passing includes message aggregation and node update. During message aggregation, each node collects and aggregates messages from its neighbors. During node update, the aggregated message is combined with the current representation of the node to update the feature vector of the node.

GIN is type of GNN that utilizes an aggregation function where each node updates the features of the node by combining the current features with the features of the neighbors followed by a multi-layer perceptron (MLP). As a result, GINs are able to achieve maximum discriminative power by uniquely encoding the graph structure.

D-MPNNs are a type GNN tailored for directed graphs, where edges have a specific direction indicating a one-way relationship between nodes. D-MPNNs propagate messages along directed edges, which is crucial for accurately modeling processes where the order of interactions matters, such as chemical reactions and certain social networks. D-MPNNs handle incoming and outgoing messages separately, incorporating edge features to capture complex dependencies, and use an update mechanism that combines the messages with the current state of the node through neural network layers.

The GNN 130 may aggregate features of a component (e.g., a component adjacent to each component or an adjacent component) connected to each component and may update (e.g., combine) features of the component by using the aggregated features of the adjacent component. When the component is a node, the component adjacent to each node may include at least one edge connected to the node or another node connected to the node through the connected edge. When the component is an edge, the component adjacent to each edge may include at least one node connected to the edge or another edge connected to the node connected to the edge.

In some embodiments, the number (e.g., N times) of aggregations and updates to be repeated in the GNN 130 are predetermined. For example, each component may be expressed by a feature that capsulizes the information of a component (e.g., an N-hop component) within an N distance from the component.

According to an embodiment, the GNN 130 may include a plurality of layers. Each layer of the GNN 130 may be classified based on an aggregation operation among operations of the GNN 130. A first layer (e.g., an input layer or a first layer 130_1) of the GNN 130 may refer to an operation from completing the calculation of features of each component from the input data 120 before performing aggregation. An intermediate layer (e.g., a hidden layer, a second layer 130_2, or an N−1th layer 130_N−1) of the GNN 130 may be referred to as an operation after performing the aggregation before performing a subsequent aggregation. A final layer (e.g., an output layer or an Nth layer 130_N) of the GNN 130 may be referred to as an operation that updates features of the component by using aggregated features of an adjacent component in each component after performing the aggregation.

In FIG. 1, for example, an electronic device may repeat N times of calculations of features and may repeat N−1 times of calculations of features and aggregations of features of an adjacent component between the calculations of features by using the GNN 130. The electronic device may obtain the input data 120 based on the input graph 110.

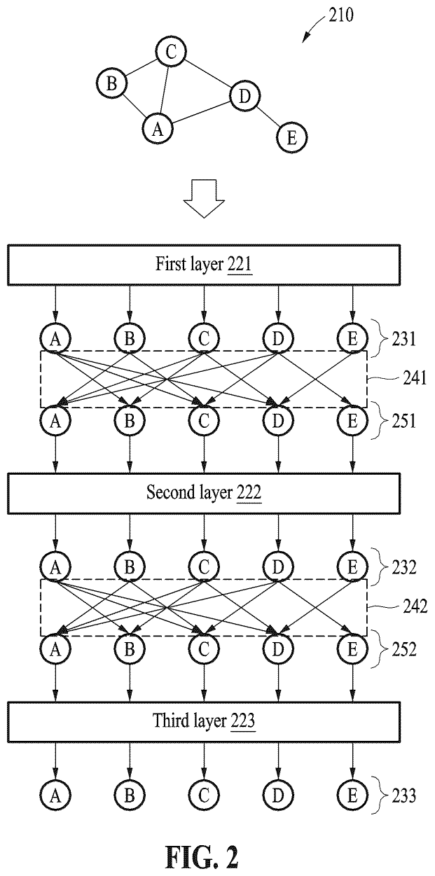

The electronic device may compute features (e.g., first features) of each component of the graph 110 by applying the first layer 130_1 to the input data 120. The electronic device may propagate the first features as a calculation result of the first layer 130_1 to adjacent components, and each node may aggregate the first feature(s) from the adjacent component(s). The electronic device may compute a second feature by combining the first features and applying the first features of the component and the first features of an adjacent component of the component, in each component to the second layer 130_2. Likewise, the electronic device may compute Nth features of components by combining N−1th features of the components computed through the N−1th layer 130_N−1 by using the Nth layer 130_N. When the GNN 130 includes N layers, the Nth features may be substantially the same as the output data 140 of the GNN 130. Further detail of the input graph 110 and the GNN 130 are described with reference to FIG. 2.

FIG. 2 illustrates an example of the input graph and the GNN according to embodiments of the present disclosure. According to an embodiment, an electronic device may compute features of components of an input graph 210 by applying the input graph 210 to a GNN. For example, the GNN of FIG. 2 may include three layers (e.g., first layer 221, second layer 222, and third layer 223). The electronic device may perform three calculations to obtain features and perform two aggregations of features through the GNN.

FIG. 2 illustrates that features of nodes are computed and/or aggregated, and features of edges might not be shown or described to be computed and/or aggregated in the GNN. However, the features of the edges of the GNN may be computed and/or aggregated as described above with reference to FIG. 1.

The electronic device may input the input graph 210 and/or input data representing the input graph 210 to the GNN. For example, the input graph 210 may include five nodes (e.g., a node A, a node B, a node C, a node D, and a node E) and may be an undirected graph including six edges (e.g., an edge AB, an edge AC, an edge AD, an edge BC, an edge CD, and an edge DE). The electronic device may obtain first features 231 by applying a first layer 221 to the input graph 210 or the input data. The first features 231 may include, for example, a first feature of the node A, a first feature of the node B, a first feature of the node C, a first feature of the node D, and a first feature of the node E.

In operation 241, the electronic device may relay (e.g., aggregate) a first feature of each node to an adjacent node based on a connectivity relation of the input graph 210. The electronic device may aggregate first features of nodes adjacent to the node for each node. In FIG. 2, aggregated first features 251 may be generated by aggregating the first features of nodes (e.g., the nodes B, C, and D) adjacent to the node (e.g., the node A) for each node (e.g., the node A). For example, the electronic device may aggregate the first features of the nodes B, C, and D for the node A based on the connectivity relation depicted in input graph 210. For example, the electronic device may aggregate the first features of the nodes A and C for the node B. For example, the electronic device may aggregate the first features of the nodes A, B, and D for the node C. For example, the electronic device may aggregate the first features of the nodes A, C, and E for the node D. For example, the electronic device may aggregate the first features of the node D for the node E.

Then, the electronic device may compute a second feature 232 of the node by applying a second layer 222 to the aggregated first features 251 for each node from the input graph 210. The electronic device may compute the second features 232 for each of nodes.

In operation 242, the electronic device may relay (e.g., aggregate) a second feature of each node to an adjacent node based on a connectivity relation of the input graph 210. The electronic device may aggregate the second features 232 of nodes adjacent to the node for each node. Similar to operation 241, aggregated second features 252 may be generated by aggregating the second features of nodes (e.g., the nodes B, C, and D) adjacent to the node (e.g., the node A) for each node (e.g., the node A). For example, the electronic device may aggregate the second features of the nodes B, C, and D for the node A. For example, the electronic device may aggregate the second features of the nodes A and C for the node B. For example, the electronic device may aggregate the second features of the nodes A, B, and D for the node C. For example, the electronic device may aggregate the second features of the nodes A, C, and E for the node D. For example, the electronic device may aggregate the second features of the node D for the node E.

The electronic device may compute a third feature 233 of the node by applying a third layer 223 to the aggregated second features 252 for each node. The electronic device may compute the third features 233 for the plurality of nodes and may obtain the third features 233 as output data of the GNN.

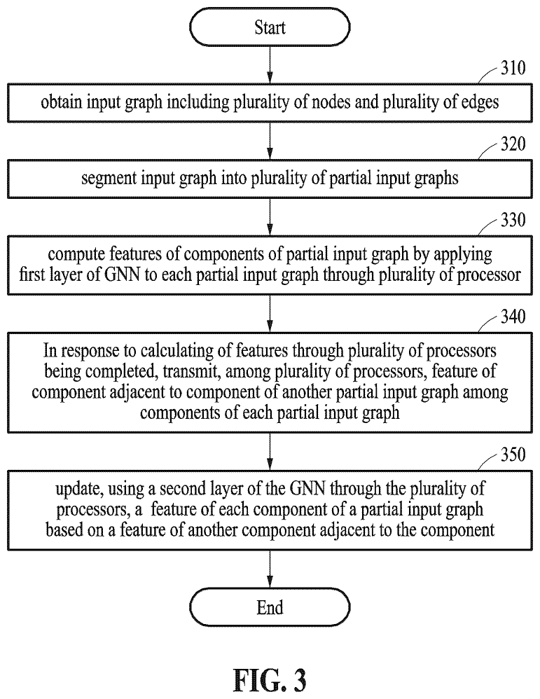

FIG. 3 illustrates an example of a method for applying a GNN to an input graph to a GNN using an electronic device according to embodiments of the present disclosure. In some examples, these operations are performed by an electronic device including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations

According to an embodiment, an electronic device may include a plurality of processors. In some embodiments of the present disclosure, the electronic device including the plurality of processors may be referred to as operating a multi-processor. The electronic device may perform an operation of applying the GNN to the input graph through parallel state processing using the plurality of processors.

At operation 310, the electronic device may obtain the input graph including a plurality of nodes and a plurality of edges. The input graph may refer to a graph representing a target of analysis. The target of analysis may include a plurality of entities. Each node may represent a corresponding entity. Each edge may represent a relation between entities. Each node may have the information of the node. Each edge may have the information of how each node interacts with another node in the input graph.

According to an embodiment, the target of analysis may be a matter. The matter may include a plurality of atoms. The plurality of atoms may be positioned at a certain distance. Alternatively, a bond (e.g., an ionic bond, a covalent bond, or a metallic bond) between two atoms may be established.

For example, a node of the input graph may correspond to an atom included in the matter. An edge of the input graph may correspond to at least one of a distance or bond between atoms. For example, a distance between a first atom and a second atom is less than or equal to a threshold distance, there may be an edge between a first node corresponding to the first atom and a second node corresponding to the second atom. For example, when a bond is established between the first atom and the second atom, there may be an edge between the first node corresponding to the first atom and the second node corresponding to the second atom.

The information of a node corresponding to an atom may include the atomic number, position, ionization information of the atom, or a combination thereof. The information of an edge may include a distance value between atoms, a type of bond between the atoms, the strength of the bond between the atoms, or a combination thereof.

At operation 320, the electronic device may segment the input graph into a plurality of partial input graphs. For example, the electronic device may segment the components (e.g., the nodes or the edges) of the input graph into the plurality of partial input graphs. Each partial input graph may include some of the components of the input graph. Some components (e.g., a node) among components of each partial input graph may be adjacent to a component (e.g., a node) of another partial input graph. The electronic device may store a connectivity relation between partial input graphs. As described below, each partial input graph may be applied to one or more layers of the GNN, and an operation on each partial input graph may be performed by one processor. Further detail on segmenting the input graph into the plurality of partial input graphs is described with reference to FIG. 3.

At operation 330, the electronic device may compute features of components of a partial input graph by applying a first layer of the GNN to each partial input graph through the plurality of processors. The plurality of processors may respectively correspond to the plurality of partial input graphs. Each processor may compute features of components of a partial input graph by applying the partial input graph corresponding to the processor to a first layer of the GNN.

According to an embodiment, the electronic device may segment the input graph into a number of partial input graphs, in which the number of partial input graphs is the same as the number (or the number of processors available for the processing of the input graph) of processors included in the electronic device. For example, the electronic device may include a first processor and a second processor. The electronic device may segment the input graph into a first partial input graph and a second partial input graph.

According to an embodiment, the electronic device may compute, in parallel state, features of components of the second partial input graph through the second processor when features of components of the first partial input graph are computed through the first processor between the plurality of processors. The electronic device may process the input graph by applying, in parallel state, the input graph to the GNN by using the plurality of processors. Accordingly, the processing speed can be reduced.

According to an embodiment, the electronic device may obtain the input data from each partial input graph. The input data may be data including the information of a node and an edge included in a partial input graph. In some cases, the input data may refer to a result of converting the partial input graph into an input format of the GNN. The electronic device may apply input data obtained from the partial input graph to the GNN. The electronic device may compute features of components (e.g., a node or an edge) of the partial input graph by applying the input data to the first layer of the GNN. The input data applied to the first layer may also be referred to as first input data.

At operation 340, in response to the computing of the features through the plurality of processors being completed, the electronic device may transmit, among the plurality of processors, a feature of a component adjacent to a component of another partial input graph among components of each partial input graph. For example, after completing the application (the calculation of the features) of the first layer of the GNN, the electronic device may perform aggregation of the computed features among the plurality of processors through the first layer.

As described above with reference to FIGS. 1 and 2, the features, after being computed, may be updated based on features of aggregated features of an adjacent component in the GNN. Since the electronic device performs the calculation of the features through the plurality of processors, one processor may receive features computed by another processor to aggregate features of an adjacent component. For example, when the processor has completed the calculation of the features, the calculation of features of the other processor might not been completed (e.g., when the other processor is computing the features), the processor may wait for the calculation of the features through the other processor to aggregate the features computed by the other processor.

When the calculation of features through some processors among the plurality of processors is completed, the electronic device may wait until the calculation of the features through the remaining processors among the plurality of processors is completed. When the calculation of the features through the first processor among the plurality of processors is completed, based on the connectivity relation between partial input graphs, the electronic device may wait for the completion of feature calculation of the second partial input graph adjacent to the first partial input graph corresponding to the first processor.

The electronic device may transmit, among the plurality of processors, features of components adjacent to components of another partial input graph among components of each partial input graph. Each of the plurality of processors may include a communication module (e.g., a communication circuit) corresponding to the processor, and each processor may transmit or receive a signal to or from another processor.

In some embodiments of the present disclosure, the example of the electronic device transmitting features of components adjacent to components of another partial input graph among components of each partial input graph is mainly described, but examples are not necessarily limited thereto. According to an embodiment, the electronic device may transmit features of components including adjacent components. For example, among components of each partial input graph, the electronic device may transmit features of other components (e.g., non-adjacent components) other than components adjacent to components of another partial input graph. For example, each processor of the electronic device may transmit features of all components of each partial input graph corresponding to the processor to another processor and/or may receive features of all components of a partial input graph corresponding to the other processor from the other processor.

At operation 350, the electronic device may update, using a second layer of the GNN through the plurality of processors, a feature of each component of a partial input graph based on a feature of another component adjacent to the component. For example, each processor may apply the second layer of the GNN to components of a partial input graph corresponding to the processor and features of components adjacent to the components. The second layer may include an operation of updating features of each component based on features of another component adjacent to the component.

For example, the first node of the input graph may be included in the first partial input graph and the second node of the input graph may be included in the second partial input graph. The first and second nodes of the input graph may be connected through an edge. For example, the first node of the first partial input graph may be adjacent to the second node of the second partial input graph.

The first processor may compute features of the first node by applying the first layer of the GNN to the first partial input graph. In parallel state, the second processor may compute features of the second node by applying the second layer of the GNN to the second partial input graph. Based on the completion of feature calculation through the first and second processors, the electronic device may relay the features of the first node from the first processor to the second processor and may relay the features of the second node from the second processor to the first processor.

In some cases, the first processor may use the features of the second node, which are received from the second processor, to update the features of the first node. In some cases, the second processor may use the features of the first node, which are received from the first processor, to update the features of the second node. For example, the first processor may update the features of the first node by combining the features of the second node to the first layer as features of a node adjacent to the first node. The second processor may update the features of the second node by combining the features of the first node to the second layer as features of a node adjacent to the second node.

According to an embodiment, the input graph may represent the matter, a node of the input graph may correspond to an atom, and an edge of the input graph may correspond to a distance or bond between atoms. The input data may include at least one of the atomic number, position, or ionization information of each of the atoms corresponding to the plurality of nodes. The electronic device may compute features by applying the GNN to the input data (or partial input data). The features may represent at least one of the energy, force, or stress of atoms. Based on the computed features, the electronic device may generate a prediction of a result of a chemical reaction between atoms, a position change of the atoms over time, or a combination thereof.

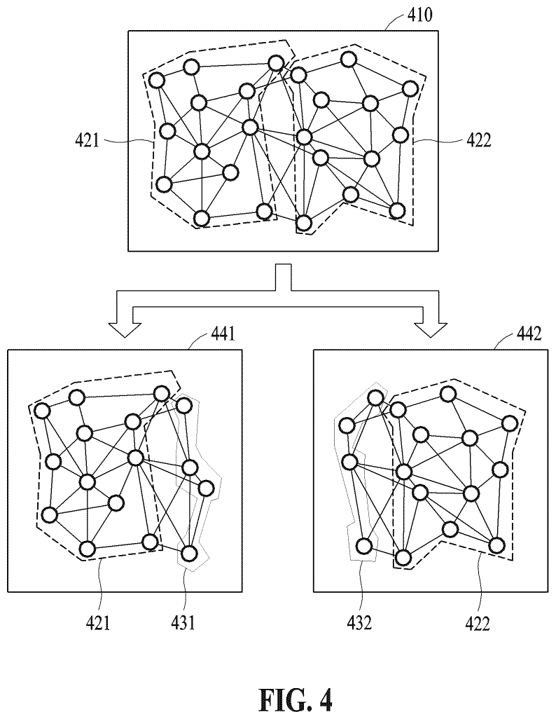

FIG. 4 illustrates an example of an operation of the electronic device segmenting an input graph into a plurality of partial input graphs according to embodiments of the present disclosure. The example shown includes input graph 410, first partial input graph 441, second partial input graph 442. In one aspect, the input graph 410 includes first node group 421 and a second node group 422. In one aspect, the first partial input graph 441 includes first node group 421 and first additional node group 431. In one aspect, the second partial input graph 442 includes second node group 422 and second additional node group 432.

According to an embodiment, an electronic device may segment an input graph 410 into a plurality of partial input graphs. The electronic device may segment a plurality of components of the input graph 410 into a plurality of component groups. Hereinafter, for ease of description, the disclosure describes an embodiment of segmenting the input graph 410 into two partial input graphs, but examples are not necessarily limited thereto. For example, the input graph 410 may be segmented into three or more partial input graphs.

The electronic device may segment the plurality of components of the input graph 410 into a first component group and a second component group. The electronic device may segment components, where the features of the segmented components are computed among the plurality of components of the input graph 410. For example, when features of a node of the input graph 410 are computed, and features of an edge of the input graph 410 might not be computed, then the input graph 410 may be segmented based on the node. When the features of a node of the input graph 410 might not be computed, and the features of an edge of the input graph 410 are computed, then the input graph 410 may be segmented based on the edge. When the features of a node of the input graph 410 are computed and the features of an edge of the input graph 410 are computed, then the input graph 410 may be segmented based on the node and the edge. In FIG. 4, for ease of description, the disclosure describes an embodiment of the features of a node may be computed and the features of an edge might not be computed. The electronic device may segment a plurality of nodes of the input graph 410 into a first node group 421 and a second node group 422.

According to an embodiment, the electronic device may classify the plurality of components such that a difference between the first number of components belonging to the first component group and the second number of components belonging to the second component group is less than or equal to a threshold number. For example, the electronic device may segment the plurality of nodes such that a difference between the first number of nodes belonging to the first node group 421 and the second number of nodes belonging to the second node group 422 is less than or equal to a threshold number.

The electronic device may classify the plurality of nodes of the input graph 410 into the first node group 421 and the second node group 422 based on differences in the number of nodes and/or edges. The node number difference may be a difference between the number of first nodes belonging to the first node group 421 and the number of second nodes belonging to the second node group 422. In some cases, the edge number difference may be a difference between the number of first edges between the nodes belonging to the first node group 421 and the number of second edges between the nodes belonging to the second node group 422. For example, the electronic device may classify the plurality of nodes of the input graph 410 into node groups such that the node number difference and/or the edge number difference may be minimized. For example, the first node group 421 may include 8 nodes and the second node group 422 may include 9 nodes.

According to an embodiment, the electronic device may determine an operation amount to be applied to a component based on the number of other components adjacent to the component and operation information of an operation to be applied to the component for each of the plurality of components. For example, for each of the plurality of nodes, the electronic device may determine an operation amount to be applied to the node based on the number (e.g., the number of edges connected to the node) of nodes adjacent to the node and operation information of an operation to be applied to the node. For example, when a node has three adjacent nodes, the operation amount may be three. In some cases, the operation amount refers to the number of operations or steps to be performed to obtain a result.

The electronic device may classify a plurality of components of the input graph 410 into component groups based on a difference between cumulative operation amounts of components belonging to each component group. For example, the electronic device may classify the plurality of components such that a difference between a first cumulative operation amount and a second cumulative operation amount is less than or equal to a threshold operation amount. For example, the first cumulative operation amount is determined based on the nodes belonging to the first node group 421 and edges between the nodes. For example, the second cumulative operation amount is determined based on the nodes belonging to the second node group 422 and edges between the nodes.

After obtaining a plurality of component groups, the electronic device may generate an additional component group, where the additional component group includes nodes from a node group that overlap with another component group. For example, the additional component group maintain a relation among the plurality of component groups as shown in the input graph 410,

For example, the electronic device, among the plurality of nodes of the input graph 410, may determine a first additional node group 431 including nodes that belong to the second node group 422 and are adjacent to a node of the first node group 421. For example, the electronic device may determine a second additional node group 432 including nodes that belong to the first node group 421 and are adjacent to a node of the second node group 422. In some embodiments of the present disclosure, an additional node group (e.g., the first additional node group 431 or the second additional node group 432) may be referred to as a dummy node group. In some cases, the first additional node group 431 or the second additional node group 432 may include substantially the same number of nodes and edges, configurations, data structure, information, or a combination thereof.

The electronic device may generate a first partial input graph 441 based on the first node group 421 and the first additional node group 431. The electronic device may generate a second partial input graph 442 based on the second node group 422 and the second additional node group 432.

Components of an additional node group may have a formal data structure for inputting features of components adjacent to components of a node group. In some cases, features of the components of the additional node group may be updated by a processor different from a processor corresponding to the partial input graph including the additional node group.

For example, a first processor may correspond to the first partial input graph 441 and a second processor may correspond to a second partial input graph 442. The first processor may update features of a node of the first partial input graph 441 by using features of a node adjacent to the node. In some cases, features of the first partial input graph 441 may include features of nodes adjacent to nodes of the first node group 421 but might not include nodes adjacent to nodes of the first additional node group 431. Accordingly, the first processor may update features of nodes of the first node group 421 of the first partial input graph 441 and might not readily update features of nodes of the first additional node group 431. In some cases, the features of the nodes of the first additional node group 431 may be updated by the second processor through the second partial input graph 442.

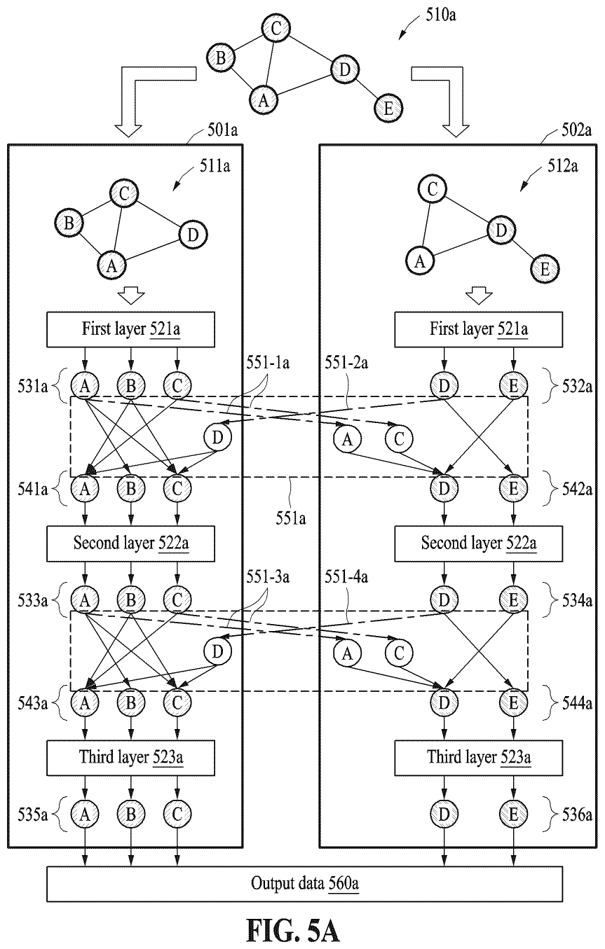

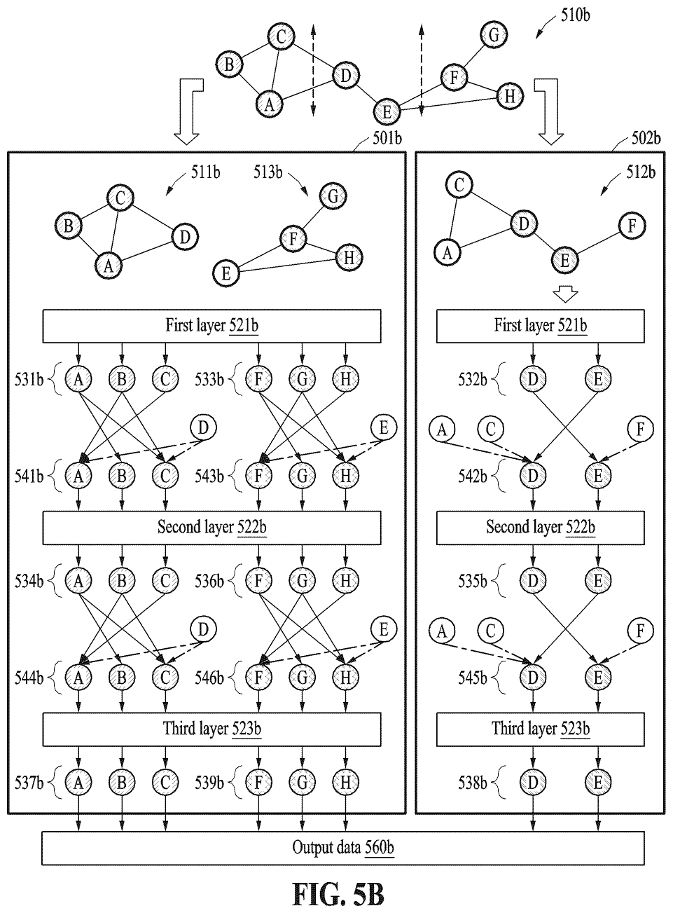

As described below with reference to FIGS. 5A and 5B, the features of the nodes of the first additional node group 431 may be computed by the second processor. The feature of the nodes of the first additional node group 431 may be relayed from the second processor to the first processor. Likewise, features of the nodes of the second additional node group 432 may be computed by the first processor. Then, the features of the nodes of the second additional node group 432 may be relayed from the first processor to the second processor.

FIG. 5A illustrates an example of an operation of the electronic device applying a plurality of partial input graphs, in parallel state, to a GNN according to embodiments of the present disclosure. According to an embodiment, an electronic device may apply a plurality of partial input graphs segmented from an input graph 510a, in parallel state, to the GNN.

The electronic device may obtain the input graph 510a. The input graph 510a may include five nodes (e.g., a node A, a node B, a node C, a node D, and a node E) and six edges (e.g., an edge AB, an edge AC, an edge AD, an edge BC, an edge CD, and an edge DE). In FIG. 4, for ease of description, the present disclosure describes an embodiment of features of a node that is computed and features of an edge might not be computed, but examples are not necessarily limited thereto. As described above, the electronic device according to some embodiments may compute at least one of the features of a node, the features of an edge, or features of a node and an edge according to the input graph 510a and/or the GNN.

The electronic device may segment the input graph 510a into a first partial input graph 511a and a second partial input graph 512a. In FIG. 5A, for example, the electronic device may classify five nodes of the input graph 510a into a first node group including the nodes A, B, and C and a second node group including the nodes D and E. The electronic device may determine a first additional node group including the node D connected to the first node group and a second additional node group including the nodes A and C connected to the second node group. The electronic device may obtain the first partial input graph 511a based on the first node group (e.g., the nodes A, B, and C) and the first additional node group (e.g., the node D). The electronic device may obtain the second partial input graph 512a based on the second node group (e.g., the nodes D and E) and the second additional node group (e.g., the nodes A and C).

In FIG. 5A, operation 501a may be an operation performed by a first processor, operation 502a may be an operation performed by a second processor, and operations 501a and 502a may be performed in parallel state at least partially. For example, the first processor may correspond to the first partial input graph 511a and the second processor may correspond to the second partial input graph 512a.

A plurality of processors of the electronic device may compute first features by applying a first layer 521a of the GNN to each partial input graph. For example, the first processor of the electronic device may compute first features 531a of nodes (e.g., the nodes A, B, and C) of the first node group of the first partial input graph 511a by applying the first layer 521a of the GNN to the first partial input graph 511a. For example, the second processor of the electronic device may compute first features 532a of nodes (e.g., the nodes D and E) of the second node group of the second partial input graph 512a by applying the first layer 521a of the GNN to the second partial input graph 512a.

As described above with reference to FIG. 3, the calculation of the first features 531a of the first node group through the first processor of the electronic device may be performed in parallel state to the calculation of the first features 532a of the second node group through the second processor of the electronic device. For example, the electronic device can simultaneously compute the first features 531a of the first node group using the first processor and the second features 532a of the second node group using the second processor. Accordingly, the processing speed can be reduced.

In operation 551a, the first and second processors of the electronic device may transmit one or more features of the first features 531a and 532a. For example, the electronic device may determine a target component adjacent to one partial input graph (e.g., the second partial input graph 512a) among components of another partial input graph (e.g., the first partial input graph 511a) based on a connectivity relation of the input graph 510a. For example, the electronic device may determine a node (e.g., the node A and/or C) adjacent to the first partial input graph 511a among the nodes of the second partial input graph 512a to be a target node. For example, the electronic device may determine a node (e.g., the node D) adjacent to the second partial input graph 512a among the nodes of the first partial input graph 511a to be a target node.

The electronic device may relay features of a target component computed by each processor from the processor to another processor. For example, in operation 551-1a, the first processor may transmit features of a target component (e.g., the node A and/or C) computed by the first processor to the second processor. The second processor may receive the features of the target component (e.g., the node A and/or C) computed by the first processor from the first processor. The second processor relays the features of the target component (e.g., the node A and/or C) computed by the first processor to the node (e.g., node D) of the second partial input graph 512a based on the connectivity relation.

For example, in operation 551-2a, the second processor may transmit features of a target component (e.g., the node D) computed by the second processor to the first processor. The first processor may receive the features of the target component (e.g., the node D) computed by the second processor from the second processor. The first processor relays the features of the target component (e.g., the node D) computed by the second processor to the node (e.g., node A and/or C) of the first partial input graph 511a based on the connectivity relation.

The electronic device may aggregate features of a node adjacent to each node through the plurality of processors (e.g., the first processor and the second processor). For example, the first processor may aggregate features of the node D computed by the second processor through the first layer 521a with features of the node A, features of the node B, and features of the node C computed by the first processor through the first layer 521a. For example, based on the connectivity relation depicted in first partial input graph 511a, features of the node D is combined with features of the node A and features of node C, independently. For example, features of node D is combined with features of node A, and features of node D is combined with features of node C. In some cases, aggregated features 541a for each node (e.g., nodes A, B, and C) are generated based on the corresponding features of adjacent nodes generated by the first layer 521a using the first processor and the second processor.

For example, the second processor may aggregate the features of the node A and the features of the node C, which are computed by the first processor through the first layer 521a, with the features of the node D and features of the node E, which are computed by the second processor through the first layer 521a. For example, based on the connectivity relation depicted in second partial input graph 512a, features of the nodes A and C are combined with features of the node D. In some cases, aggregated features 542a for each node (e.g., nodes D and E) are generated based on the corresponding features of adjacent nodes generated by the first layer 521a using the first processor and the second processor.

The electronic device may compute second features updated from the first features by using a second layer 522a of the GNN. Each processor may compute the second features by applying the second layer 522a to aggregated features for a partial input graph corresponding to the processor. For example, the first processor may update features of the first partial input graph 511a by applying the second layer 522a to the aggregated features 541a for each node of the first node group in the first partial input graph 511a. The first processor may compute second features 533a based on a result obtained from applying the second layer 522a to the aggregated features 541a. For example, the second processor may update features of the second partial input graph 512a by applying the second layer 522a to the aggregated features 542a for each node of the second node group in second partial input graph 512a. The second processor may compute second features 534a based on a result obtained from applying the second layer 522a to the aggregated features 542a.

The electronic device may relay features of a target component computed by each processor from the processor to another processor. After computing the second features based on a connectivity relation among the plurality of partial input graphs, the electronic device may transmit one or more features of the second features among the plurality of processors.

For example, like operations 551-1a and 551-2a, in operation 551-3a, the features of the node A and the features of the node C are relayed from the first processor to the second processor, and, in operation 551-4a, the features of the node D may be relayed from the second processor to the first processor.

Each processor of the electronic device may compute third features (e.g., third features 535a or third features 536a) updated from the second features by applying a third layer 523a to aggregated second features (e.g., aggregated second features 543a and aggregated second features 544a) for a partial input graph using the corresponding processor. The electronic device may compute second features updated from second features computed by a processor and second features received from another processor by using each of the plurality of processors. For example, the first processor may compute third features 535a by applying the third layer 523a to the aggregated second features 543a. For example, the second processor may compute third features 536a by applying the third layer 523a to the aggregated second features 544a. In some cases, the electronic device may obtain output data 560a based on the third features 535a computed by the first processor and the third features 536a computed by the second processor.

FIG. 5B illustrates an example of an operation of the electronic device applying a plurality of partial input graphs, in parallel state, to a GNN according to embodiments of the present disclosure. In some embodiments, the number of the plurality of partial input graphs is greater than the number of processors.

According to an embodiment, an electronic device may provide a plurality of partial input graphs segmented from an input graph 510b, in parallel state, to the GNN. When the number of the plurality of partial input graphs is greater than the number of processors, two or more partial input graphs may be assigned to one processor. For example, two or more independent partial input graphs (e.g., when components of one partial input graph are not adjacent to components of another partial input graph) among the plurality of partial input graphs may be assigned to one processor and may be provided, in parallel state, to the GNN.

The electronic device may obtain an input graph 510b. The input graph 510b may include eight nodes (e.g., a node A, a node B, a node C, a node D, a node E, a node F, a node G, and a node H) and ten edges (e.g., an edge AB, an edge AC, an edge AD, an edge BC, an edge CD, an edge DE, an edge EF, an edge EH, an edge FG, and an edge FH). In FIG. 4, for ease of description, the present disclosure describes an embodiment of features of a node that is computed and features of an edge might not be computed, but examples are not necessarily limited thereto. As described above, the electronic device according to some embodiments may compute at least one of the features of a node or the features of an edge according to the input graph 510b and/or the GNN.

The electronic device may segment the input graph 510b into a first partial input graph 511b, a second partial input graph 512b, and a third partial input graph 513b. In FIG. 5B, for example, the electronic device may classify eight nodes of the input graph 510b into a first node group including the nodes A, B, and C, a second node group including the nodes D and E, and a third node group including the nodes F, G, and H. The electronic device may determine a first additional node group, a second additional node group, and a third additional node group. The first additional node group may include the node D connected to the first node group. The second additional node group may include the nodes A and C connected to the one side of second node group and the node F connected to the opposite side of the second node group. The third additional node group may include the node E connected to the third node group. In some cases, the data structure of between each of the additional node group and the corresponding node group is substantially the same as the data structure of a portion of the input graph 510b.

The electronic device may obtain the first partial input graph 511b based on the first node group (e.g., the nodes A, B, and C) and the first additional node group (e.g., the node D). The electronic device may obtain the second partial input graph 512b based on the second node group (e.g., the nodes D and E) and the second additional node group (e.g., the nodes A, C, and F). The electronic device may obtain the third partial input graph 513b based on the third node group (e.g., the nodes F, G, and H) and the third additional node group (e.g., the node E).

The electronic device may determine a correspondence between processors and partial input graphs based on the number (e.g., 2) of available processors and the number (e.g., 3) of partial input graphs. The electronic device may determine partial input graphs (e.g., a pair of partial input graphs) in which all components of one partial input graph are not adjacent to components of another partial input graph among partial input graphs. In FIG. 5B, for example, since components of the first partial input graph 511b are not adjacent to components of the third partial input graph 513b, the electronic device may determine the first partial input graph 511b and the third partial input graph 513b to be independent partial input graphs. Accordingly, the electronic device may assign the first partial input graph 511b and the third partial input graph 513b to the first processor and may assign the second partial input graph 512b to the second processor, or vice versa.

In FIG. 5B, operation 501b may be an operation performed by the first processor, operation 502b may be an operation performed by the second processor. In some cases, operations 501b and 502b may be performed in partially in a parallel state. The first processor may correspond to the first partial input graph 511b and the third partial input graph 513b and the second processor may correspond to the second partial input graph 512b.

The plurality of processors of the electronic device may compute first features by applying the first layer 521a of the GNN to each partial input graph. For example, the first processor of the electronic device may compute the first features 531b of nodes (e.g., the nodes A, B, and C) of the first node group of the first partial input graph 511b by applying the first layer 521b of the GNN to the first partial input graph 511b. The first processor of the electronic device may compute first features 533b of nodes (e.g., the nodes F, G, and H) of the third node group of the third partial input graph 513b by applying the first layer 521b of the GNN to the third partial input graph 513b. For example, the second processor of the electronic device may compute first features 532b of nodes (e.g., the nodes D and E) of the second node group of the second partial input graph 512b by applying the first layer 521b of the GNN to the second partial input graph 512b.

The calculation of the first features 531a and 533b of the first and third node groups, respectively, through the first processor of the electronic device may be performed in a parallel state to the calculation of the first features 532b of the second node group through the second processor of the electronic device. For example, in the parallel state, the first processor and the second processor simultaneously computes the first features of the corresponding components (e.g., nodes, edges, or a combination thereof).

The first and second processors of the electronic device may transmit one or more features of the first features 531b and 533b and first features 532b to each other. For example, the electronic device may determine a target component adjacent to one partial input graph (e.g., the second partial input graph 512b) among components of another partial input graph (e.g., the first partial input graph 511b) based on a connectivity relation of the input graph 510b. For example, the electronic device may determine a node (e.g., the node A and C or F) adjacent to the first partial input graph 511b or the third partial input graph 513b, respectively, among the nodes of the second partial input graph 512b to be a target node. For example, the electronic device may determine a node (e.g., the node D or E) adjacent to the second partial input graph 512b among the nodes of the first partial input graph 511b and the third partial input graph 513b to be a target node.

The electronic device may relay features of a target component computed by each processor from the processor to another processor. In FIG. 5B, arrows indicating the relay of features between processors may be omitted for brevity of representation. For example, the first processor may transmit features of a target component (e.g., the node A, C and/or F) computed by the first processor to the second processor. The second processor may receive the features of the target component (e.g., the node A, C, and/or F) computed by the first processor from the first processor. For example, the second processor may receive the features of the target component for nodes A and C and the features of the target component for node F to combine with features of the nodes D and E, respectively, based on the connectivity relation of the input graph 510b.

For example, the second processor may transmit features of a target component (e.g., the node D or E) computed by the second processor to the first processor. The first processor may receive the features of the target component (e.g., the node D or E) computed by the second processor from the second processor. In some cases, the first processor may combine the features of the target component of nodes D to the nodes A and C based on the connectivity relation of the input graph 510b. In some cases, the first processor may combine the features of the target component of nodes E to the nodes F and H based on the connectivity relation of the input graph 510b.

The electronic device may aggregate features of a node adjacent to each node through the plurality of processors (e.g., the first processor and the second processor). For example, the first processor may aggregate features of the node D and features of the node E, which are computed by the second processor through the first layer 521b, together with features of the node A, features of the node B, features of the node C, features of the node F, features of the node G, and features of the node H, which are computed by the first processor through the first layer 521b based on the connectivity relation depicted in input graph 510b.

For example, the second processor may aggregate the features of the node A and the features of the node C, and the features of the node F, which are computed by the first processor through the first layer 521b, with the features of the node D and features of the node E, respectively, which are computed by the second processor through the first layer 521b based on the connectivity relation depicted in input graph 510b.

The electronic device may compute second features updated from the first features by using a second layer 522b of the GNN. Each processor may compute the second features by applying the second layer 522b to aggregated features for the partial input graph corresponding to the processor. For example, the first processor may update the features of the first partial input graph 511b by applying the second layer 522b to aggregated features 541b for each node of the first node group in first partial input graph 511b. The first processor may compute second features 534b based on a result obtained from applying the second layer 522b to the aggregated features 541b.

For example, the first processor may update the features of the third partial input graph 513b by applying the second layer 522b to aggregated features 543b for each node of the third node group in third partial input graph 513b. The first processor may compute second features 536b based on a result obtained from applying the second layer 522b to the aggregated features 543b.

For example, the second processor may update features of the second partial input graph 512b by applying the second layer 522b to aggregated features 542b for each node of the second node group in second partial input graph 512b. The second processor may compute second features 535b based on a result obtained from applying the second layer 522b to the aggregated features 542b.

The electronic device may relay features of a target component computed by each processor from the processor to another processor. After computing the second features based on a connectivity relation among the plurality of partial input graphs, the electronic device may transmit one or more features of the second features among the plurality of processors. For example, the features of the node A, the features of the node C, and the features of the node F are relayed from the first processor to the second processor and the features of the node D and the features of the node E may be relayed from the second processor to the first processor.

Each processor of the electronic device may compute third features updated from the second features by applying a third layer 523b to aggregated second features (e.g., aggregated second features 544b for the first partial input graph 511b, aggregated second features 545b for the second partial input graph 512b, and aggregated second features 546b for the third partial input graph 513b) for the partial input graphs corresponding to the processors. The electronic device may compute third features updated from second features computed by a processor and second features received from another processor by using each of the plurality of processors.

For example, the first processor may compute third features 537b by applying the third layer 523b to the aggregated second features 544b for the first partial input graph 511b. For example, the first processor may compute third features 539b by applying the third layer 523b to the aggregated second features 546b the third partial input graph 513b. For example, the second processor may compute the third features 538b by applying the third layer 523b to the aggregated second features 545b for the second partial input graph 512b. In some cases, the electronic device may obtain output data 560b based on the third features 537b and 539b computed by the first processor and the third features 538b computed by the second processor.

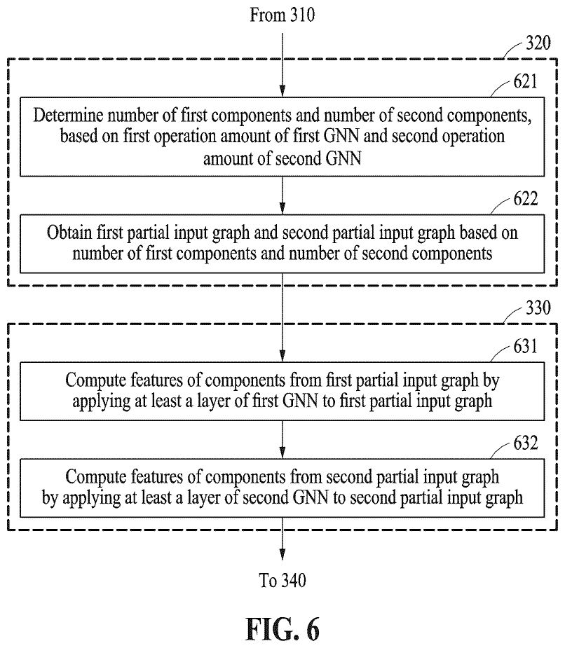

FIG. 6 illustrates an example of a method for a plurality of processors of the electronic device employing GNNs having different operation amounts according to embodiments of the present disclosure. In some examples, these operations are performed by an electronic device including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

According to an embodiment, the electronic device may apply the GNNs to a plurality of partial input graphs segmented from an input graph by using the plurality of processors. According to an embodiment, the electronic device may apply different GNNs to each partial input graph. In some cases, the plurality of GNNs applied to the partial input graphs may have different operation amounts.

At operation 621, the electronic device may determine the number of first components and the number of second components based on a first operation amount of a first GNN and a second operation amount of a second GNN. For example, the first GNN may be a GNN applied to a first partial input graph by a first processor. The second GNN may be a GNN applied to a second partial input graph by a second processor. In some embodiments, the first GNN and the second GNN may be different GNNs. For example, the first GNN and the second GNN may have different operation amounts.

The electronic device may determine the number of first components and the number of second components such that a ratio of the number of second components to the number of first components may decrease as a ratio of the second operation amount to the first operation amount increases. According to an embodiment, the electronic device may control a total operation amount (or a total operation time) performed by the first processor and a total operation amount (or a total operation time) performed by the second processor to be substantially the same by modifying or adjusting the number of first components to be less than the number of second components as the first operation amount is greater than the second operation amount. For example, if the first operation amount is 10 and the second operation amount is 5, then the electronic device may modify and set the number of first component to be 5 and the number of second component to be 10.

At operation 622, the electronic device may obtain a first partial input graph and a second partial input graph based on the number of first components and the number of second components. The number of components (e.g., the number of first components and the number of second components) may include at least one of the number of nodes, the number of edges, or the total number of nodes and edges.

For example, the electronic device may segment a plurality of components of the input graph into a first component group and a second component group. The first component group may include a number of first components (or the number of which a difference with the number of first components is less than or equal to a threshold number) and the second component group may include a number of second components (or the number of which a difference with the number of second components is less than or equal to the threshold number).

The electronic device may obtain a first partial input graph and a second partial input graph based on the first component group and the second component group. The operation of obtaining the first partial input graph and the second partial input graph may be substantially the same as the operation of segmenting the input graph into the plurality of partial input graphs as described above with reference to FIG. 4. By determining the first partial input graph and the second partial input graph based on the ratio of the number of first components and the number of second components, the electronic device is able to optimize the computational resource to generate an output data using the GNN.

At operation 631, the electronic device may compute features of components from the first partial input graph by applying at least a layer of the first GNN to the first partial input graph. For example, the electronic device may compute first features of components of the first partial input graph by applying a first layer of the first GNN to the first partial input graph.

At operation 632, the electronic device may compute features of components included in the second partial input graph by applying at least a layer of the second GNN to the second partial input graph. For example, the electronic device may compute first features of components of the second partial input graph by applying a first layer of the second GNN to the second partial input graph.

In some embodiments, when the calculation of first features through the plurality of processors is completed, one or more features of the computed first features may be transmitted or received among the plurality of processors. For example, the plurality of processors may aggregate first features computed by each processor with first features computed by another processor. The first processor may aggregate first features computed through the first layer of the first GNN and first features computed through the first layer of the second GNN and may apply the aggregated first features to the first GNN.

According to an embodiment, a format (e.g., a dimension) of features of components computed through each layer (e.g., the first layer, a second layer, or a third layer) of the first GNN may be the same as a format (e.g., a dimension) of features of components computed through each layer (e.g., the first layer, a second layer, or a third layer) of the second GNN. For example, the dimensionality of each layer of the first GNN may be the same.

According to an embodiment, when the format (e.g., the dimension) of features of components computed through each layer of the first GNN is different from the format (e.g., the dimension) of features of components computed through each layer of the second GNN, at least one of the plurality of processors may convert a format of features computed through one GNN into a format of features computed through the other GNN.

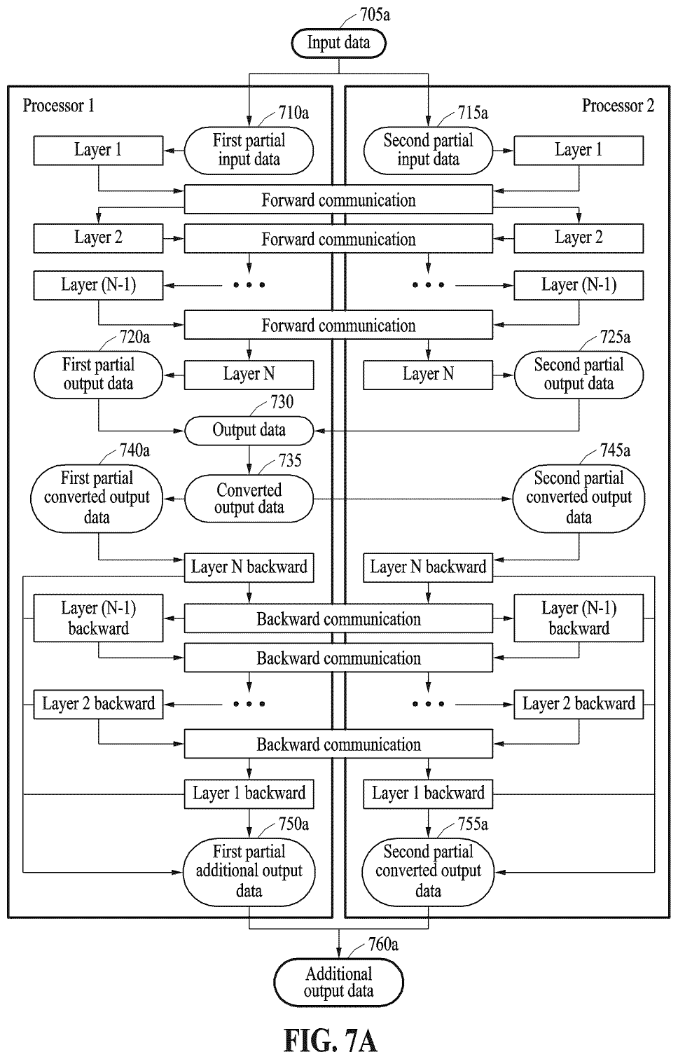

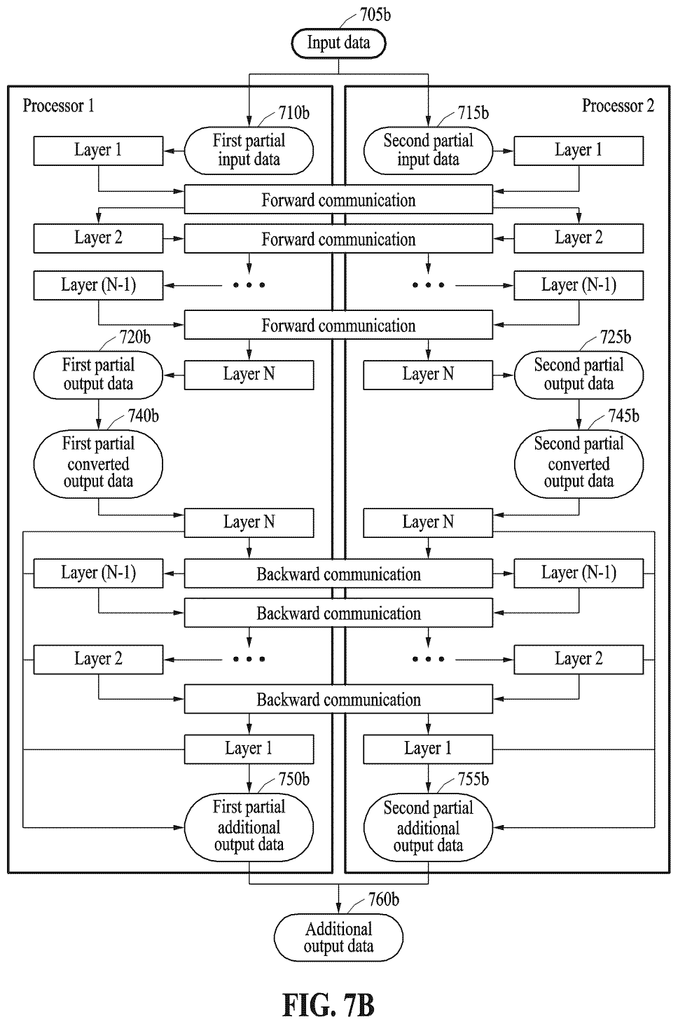

FIGS. 7A and 7B illustrate examples of an operation of the electronic device performing the forward propagation and backward propagation on a GNN according to embodiments of the present disclosure.

According to an embodiment, the electronic device may obtain output data 730 by forwardly propagating input data 705a through the GNN and may obtain additional output data 760a by backwardly propagating the output data through the GNN. The forward propagation may refer to performing an operation assigned to each layer of the GNN based on a value input to the layer and parameters (e.g., parameters included in the layer) recorded in the layer. The backward propagation may refer to computing (e.g., differentiating) a gradient for the parameters, input data, and activation values (or activation) (e.g., node features or edge features in the GNN) of each layer of the GNN.

The electronic device may apply input data 705a corresponding to a plurality of partial input graphs to an input layer (or layer 1) of the GNN. The electronic device may perform forward propagation of the input data 705a from the input layer of the GNN through a plurality of hidden layers (or intermediate layers such as layer 2 and layer N−1) of the GNN to an output layer (or layer N) of the GNN. The electronic device may obtain output data 730 from the output layer as a result of the forward propagation.

According to an embodiment, in forward propagation, each processor of the electronic device may apply the GNN to a partial input graph (or partial input data) corresponding to the processor. For example, each processor of the electronic device may perform forward propagation of the partial input data corresponding to the processor from the input layer of the GNN through the plurality of hidden layers of the GNN to an output layer of the GNN. Each processor of the electronic device may obtain partial output data (e.g., first partial output data 720a or second partial output data 725a) from a partial input graph corresponding to the processor. The electronic device may obtain output data 730 based on partial output data obtained by the plurality of processors.

The electronic device may apply the output data 730 to the output layer. The electronic device may perform backward propagation of the output data 730 from the output layer through the hidden layers to the input layer. The electronic device may obtain additional output data 760a from the input layer as a result of the backward propagation.

According to an embodiment, in backward propagation, each processor of the electronic device may apply the GNN to partial output data corresponding to the processor. For example, each processor of the electronic device may perform backward propagation of the partial output data corresponding to the processor from the output layer of the GNN through the plurality of hidden layers of the GNN to the input layer of the GNN. Each processor of the electronic device may obtain partial additional output data (e.g., first partial additional output data 750a or second partial additional output data 750b) from partial output data corresponding to the processor. The electronic device may obtain additional output data 760a based on partial additional output data obtained by the plurality of processors.

According to an embodiment, the input graph may be a graph on a matter, where each node may correspond to an atom, and each edge may correspond to at least one of a distance or bond between atoms. The input data (or partial input data) of the input graph (or a partial input graph) may include information (e.g., an atomic number, a position of an atom, or the ionization information of the atom) of a node and/or information (e.g., a distance between atoms, a relative vector between the atoms, or the type of bond between atoms) of an edge. The electronic device may obtain the output data 730 (or partial output data) by applying the GNN, using forward propagation, to the input data 705a (or partial input data). The output data 730 may include features of nodes, and features of each node may represent the energy of an atom corresponding to the node.

The electronic device may obtain the additional output data (or partial additional output data) by applying the GNN, using backward propagation, to the output data (or partial output data). The additional output data may include additional features of nodes. For example, additional features of each node may represent at least one of the force, stress, band gap, highest occupied molecular orbital (HOMO), or lowest unoccupied molecular (LUMO) of an atom corresponding to the node. In some cases, the additional feature of each node may include additional information about the node.

The additional output data may represent at least one of a gradient for the input data 705a of the output data 730 or a gradient for the parameters of each layer of a neural network. For example, the gradient for the input data 705a may represent at least one of the force or stress of an atom. For example, the gradient for the parameters of each layer of a neural network may represent an amount of change required for the training of the neural network.

Referring to FIGS. 7A and 7B, the electronic device may segment the input data 705a into first partial input data 710a and second partial input data 715a. The first partial input data 710a may be processed using a first processor (e.g., processor 1) of the electronic device and the second partial input data may be processed using a second processor (e.g., processor 2) of the electronic device. The first processor may forwardly apply the first partial input data 710a to a first layer of the GNN and the second processor may forwardly apply the second partial input data 715a to the first layer of the GNN. After the forward propagation of the first processor applied to the first layer and the forward propagation of the second processor applied to the first layer, the first processor and the second processor may transmit one or more features of results (e.g., forward propagation result) of the first layer to each other. The transmission of forward propagation results of a layer may be referred to as the forward communication. In some cases, the GNN may include N layers, and each of the first and second processors may repeat the forward propagation and forward communication for each layer. Each of the first and second processors may obtain partial output data (e.g., first partial output data 720a and second partial output data 725a) through the forward propagation of an Nth layer of the GNN.

In FIG. 7A, the electronic device may obtain the output data 730 by combining the first partial output data 720a with the second partial output data 725a. The electronic device may obtain converted output data 735 from the output data 730. In some cases, the electronic device may segment the converted output data 735 to generate the first partial converted output data 740a and the second partial converted output data 745. For example, the second processor may transmit the second partial output data 725a to the first processor. The first processor may obtain the output data 730 by combining the second partial output data 725a received from the second processor with the first partial output data 720a. The first processor may convert the output data 730 into the converted output data 735. The first processor may obtain (e.g., segment) the first partial converted output data 740a and the second partial converted output data 745a from the converted output data 735 and may transmit the second partial converted output data 745a to the second processor.

In FIG. 7B, the electronic device may obtain the first partial converted output data 740b and the second partial converted output data 745b by converting the first partial output data 720b and the second first partial output data 725b, respectively. In some cases, for example, the partial converted output data includes a converted format (or dimension) of the features for each of the first partial input data 710b or the second partial input data 715b.

In FIGS. 7A and 7B, the first processor of the electronic device may process the first partial converted output data 740a and the second processor of the electronic device may process the second partial converted output data 745a. The first processor may backwardly apply the first partial converted output data 740a to the Nth layer and, in the parallel state, the second processor may backwardly apply the second partial converted output data 745a to the Nth layer. After the backward propagation of the first processor applied to the Nth layer and the backward propagation of the second processor applied to the Nth layer, the first processor and the second processor may transmit one or more features of the backward propagation results of the Nth layer to each other. The transmission of backward propagation results of a layer may be referred to as the backward communication. Each of the first and second processors may repeat the backward propagation and the backward communication for each layer of the GNN. Each of the first and second processors may obtain partial additional output data (e.g., first partial additional output data 750a and second partial additional output data 755a) through the backward propagation of the first layer of the GNN. The electronic device may obtain additional output data 760a based on the first partial additional output data 750a and the second partial additional output data 755a.

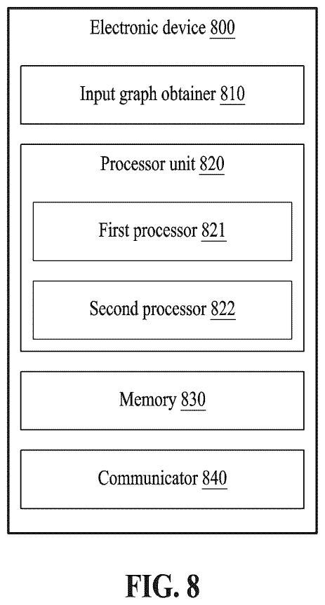

FIG. 8 illustrates an example of a configuration of the electronic device according to embodiments of the present disclosure. In some cases, the electronic device 800 may include an input graph obtainer 810, a processor unit 820, a memory 830, and a communicator 840. In one aspect, the processor unit 820 includes a first processor 821 and a second processor 822.

According to some aspects, input graph obtainer 810 is implemented as software stored in the memory 830 and executable by processor unit 820, as firmware, as one or more hardware circuits, or as a combination thereof. In one aspect, the input graph obtainer 810 may obtain an input graph. In some cases, the input graph is obtained from a database. In some cases, the input graph is provided by a user.

According to some aspects, the processor unit 820 may obtain the input graph through the input graph obtainer 810. The processor unit 820 may segment the input graph into a plurality of partial input graphs. The processor unit 820 may include a plurality of processors. According to an embodiment, the processor unit 820 may include a first processor 821 and a second processor 822. The plurality of processors may respectively correspond to a plurality of partial input graphs. In some aspects, the first processor 821 may compute features of components of a first partial input graph by applying a first layer of a GNN to the first partial input graph. In some aspects, the second processor 822 may compute features of components of a second partial input graph by applying a first layer of a GNN to the second partial input graph. In response to the completion of computing the features, the processor unit 820 may transmit one or more features among the plurality of processors. The processor unit 820 may update the features by using a second layer of the GNN.

Processor unit 820 is an intelligent hardware device, (e.g., a general-purpose processing component, a digital signal processor (DSP), a central processing unit (CPU), a graphics processing unit (GPU), a microcontroller, an application-specific integrated circuit (ASIC), a field programmable gate array (FPGA), a programmable logic device, a discrete gate or transistor logic component, a discrete hardware component, or any combination thereof). In some cases, processor unit 820 is configured to operate a memory array using a memory controller. In some cases, a memory controller is integrated into the processor unit 820. In some cases, processor unit 820 is configured to execute computer-readable instructions stored in the memory 830 to perform various functions. In some embodiments, processor unit 820 includes special-purpose components for modem processing, baseband processing, digital signal processing, or transmission processing.

According to some aspects, the memory 830 may temporarily and/or permanently store at least one of the input graph, the partial input graphs, the GNN, or the features. The memory 830 may store instructions for obtaining of the input graph, computing the features of components of the input graph, transmitting the features among the plurality of processors, and updating the features of the input graph. In some cases, the foregoing examples are just examples, and the information stored in the memory 830 is not necessarily limited thereto.

Examples of the memory 830 include random access memory (RAM), read-only memory (ROM), or a hard disk. Examples of the memory 830 include solid-state memory and a hard disk drive. In some examples, the memory 830 is used to store computer-readable, computer-executable software including instructions that, when executed, cause a processor (e.g., the processor unit 820) to perform various functions described herein.

In some cases, the memory 830 includes, among other things, a basic input/output system (BIOS) that controls basic hardware or software operations such as the interaction with peripheral components or devices. In some cases, a memory controller operates memory cells. For example, the memory controller can include a row decoder, column decoder, or both. In some cases, memory cells within the memory 830 store information in the form of a logical state.

The communicator 840 may transmit and receive at least one of the input graph, the partial input graphs, the GNN, or the features. The communicator 840 may establish a wired communication channel and/or a wireless communication channel with an external device (e.g., another electronic device and a server) and may establish communication via a long-range communication network, such as cellular communication, short-range wireless communication, local area network (LAN) communication, Bluetooth™, wireless-fidelity (Wi-Fi) direct or infrared data association (IrDA), a legacy cellular network, a fourth generation (4G) and/or 5G network, next-generation communication, the Internet, or a computer network (e.g., LAN or a wide area network (WAN)).

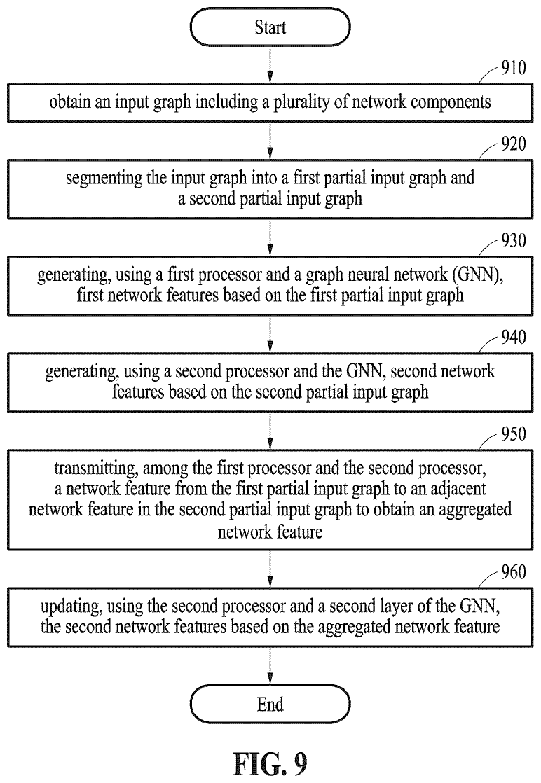

FIG. 9 illustrates an example of a method for processing graphical data using an electronic device according to embodiments of the present disclosure. In some examples, these operations are performed by an electronic device including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

At operation 910, the electronic device obtains an input graph including a plurality of network components. For example, each of the plurality of network components include a plurality of nodes and a plurality of edges. In some cases, the operations of this step refer to, or may be performed by, an input graph obtainer as described with reference to FIG. 8. Further detail on the operations of this step is described with reference to FIGS. 3-6.

At operation 920, the electronic device segments the input graph into a first partial input graph and a second partial input graph. In some cases, the operations of this step refer to, or may be performed by, a processor unit as described with reference to FIG. 8. Further detail on the operations of this step is described with reference to FIGS. 3-6.

At operation 930, the electronic device generates, using a first processor and a graph neural network (GNN), first network features based on the first partial input graph. For example, each of the first network features includes a connectivity relation between a first network component and an adjacent first network component among the plurality of network components in the input graph. In some cases, the operations of this step refer to, or may be performed by, a first processor as described with reference to FIG. 8. Further detail on the operations of this step is described with reference to FIGS. 3-6.

At operation 940, the electronic device generates, using a second processor and the GNN, second network features based on the second partial input graph. For example, each of the second network features includes a connectivity relation between a second network component and an adjacent second network component among the plurality of network components in the input graph. In some cases, the operations of this step refer to, or may be performed by, a second processor as described with reference to FIG. 8. Further detail on the operations of this step is described with reference to FIGS. 3-6.

at operation 950, the electronic device transmits, among the first processor and the second processor, a network feature from the first partial input graph to an adjacent network feature in the second partial input graph to obtain an aggregated network feature. In some cases, the operations of this step refer to, or may be performed by, a communicator as described with reference to FIG. 8. Further detail on the operations of this step is described with reference to FIGS. 3-6.

At operation 960, the electronic device updates, using the second processor and a second layer of the GNN, the second network features based on the aggregated network feature. In some cases, the operations of this step refer to, or may be performed by, a second processor as described with reference to FIG. 8. Further detail on the operations of this step is described with reference to FIGS. 3-6.

The examples described herein may be implemented by using a hardware component, a software component, and/or a combination thereof. A processing device may be implemented using one or more general-purpose or special-purpose computers, such as, for example, a processor, a controller and an arithmetic logic unit (ALU), a digital signal processor (DSP), a microcomputer, a field-programmable gate array (FPGA), a programmable logic unit (PLU), a microprocessor, or any other device capable of responding to and executing instructions in a defined manner. The processing device may run an operating system (OS) and one or more software applications that run on the OS. The processing unit also may access, store, manipulate, process, and generate data in response to execution of the software. For purpose of simplicity, the description of a processing unit is used as singular; however, one skilled in the art may appreciate that a processing unit may include multiple processing elements and multiple types of processing elements. For example, the processing unit may include a plurality of processors, or a single processor and a single controller. In addition, different processing configurations are possible, such as parallel state processors.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described examples may be recorded in non-transitory computer-readable media including program instructions to implement various operations of the above-described examples. The media may also include, alone or in combination with the program instructions, data files, data structures, and the like. The program instructions recorded on the media may be those specially designed and constructed for the purposes of examples, or program instructions may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random-access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter.

The above-described devices may act as one or more software modules in order to perform the operations of the above-described examples, or vice versa. As used herein, “A or B,” “at least one of A and B,” “at least one of A or B,” “A, B or C,” “at least one of A, B and C,” and “at least one of A, B, or C,” each of which may include any one of the items listed together in the corresponding one of the phrases, or all possible combinations thereof.

As described above, although the examples have been described with reference to the limited drawings, a person skilled in the art may apply various technical modifications and variations based thereon. For example, suitable results may be achieved if the described techniques are performed in a different order and/or if components in a described system, architecture, device, or circuit are combined in a different manner and/or replaced or supplemented by other components or their equivalents.

Therefore, the scope of the disclosure is defined not by the detailed description, but by the claims and their equivalents, and all variations within the scope of the claims and their equivalents are to be construed as being included in the disclosure.

What is claimed is:

1. A method for processing graphical data comprising:
obtaining an input graph including a plurality of network components, where each of the plurality of network components include a plurality of nodes and a plurality of edges;
segmenting the input graph into a plurality of partial input graphs, wherein the plurality of partial input graphs includes a first partial input graph and a second partial input graph;
generating, using a plurality of processors and a first layer of a graph neural network (GNN), network features based on the plurality of partial input graphs, wherein each of the network features includes a connectivity relation between a network component and an adjacent network component among the plurality of network components in the input graph;
transmitting, among the plurality of processors, a network feature from the first partial input graph to an adjacent network feature in the second partial input graph to obtain an aggregated network feature; and
updating, using the plurality of processors and a second layer of the GNN, the network features of the plurality of network components based on the aggregated network feature.

2. The method of claim 1, wherein generating the network features comprises:
computing, using a first processor of the plurality of processors, a first group of network features of the first partial input graph; and
computing, using a second processor of the plurality of processors, a second group of network features of the second partial input graph in a parallel state, wherein in the parallel state, the first processor and the second processor simultaneously computes the first group of network features and the second group of network features, respectively.

3. The method of claim 1, wherein transmitting the network feature comprises:
determining a target network component adjacent to the second partial input graph among network components of the first partial input graph based on the connectivity relation of the input graph; and
transmitting a target network feature of the target network component to a first processor corresponding to the first partial input graph through a second processor, wherein the target network feature is generated based on the second processor corresponding to the second partial input graph.

4. The method of claim 1, wherein segmenting the input graph comprises:
segmenting the plurality of nodes of the input graph into a first node group and a second node group;
determining a first additional node group including a subset of nodes from the second node group and is adjacent to a node of the first node group among the plurality of nodes of the input graph;
determining a second additional node group including a subset of nodes from the first node group and is adjacent to a node of the second node group among the plurality of nodes of the input graph;
generating the first partial input graph based on the first node group and the first additional node group; and
generating the second partial input graph based on the second node group and the second additional node group.

5. The method of claim 4, wherein segmenting the input graph comprises:
determining a first number of nodes belonging to the first node group; and
determining a second number of nodes belonging to the second node group, wherein a difference between the first number and the second number is less than or equal to a threshold value.

6. The method of claim 4, wherein segmenting the input graph comprises:
obtaining a first cumulative operation amount based on nodes belonging to the first node group and edges between the nodes; obtaining a second cumulative operation amount based on nodes belonging to the second node group and edges between the nodes; and
segmenting the plurality of network components based on the first cumulative operation amount and the second cumulative operation amount, where a difference between the first cumulative operation amount and the second cumulative operation amount is less than or equal to a threshold operation amount.

7. The method of claim 1, wherein segmenting the input graph comprises:
determining a number of first network components and a number of second network components based on a first operation amount of a first GNN and a second operation amount of a second GNN, respectively; and
obtaining the first partial input graph and the second partial input graph based on the number of first network components and the number of second network components, respectively.

8. The method of claim 1, wherein generating the network features comprises:
computing network features of network components of the first partial input graph by applying one or more layers of a first GNN to the first partial input graph; and
computing network features of network components of the second partial input graph by applying one or more layers of a second GNN to the second partial input graph.

9. The method of claim 1, further comprising:
generating first network features;
generating, using a second layer of the GNN, second network features based on the first network features and the connectivity relation among the plurality of partial input graphs;
transmitting one or more second network features to one or more processors among the plurality of processors; and
generating, using a third layer of the GNN, third network features based on the second network features and the connectivity relation among the plurality of partial input graphs.

10. The method of claim 1, wherein generating the network features comprises:
applying input data corresponding to the plurality of partial input graphs to an input layer of the GNN;
performing forward propagation using the input data;
generating output data from an output layer of the GNN based on the forward propagation;
applying the output data to the output layer of the GNN;
performing backward propagation using the output data; and
generating additional output data from the input layer of the GNN based on the backward propagation.

11. A method for processing graphical data comprising:
obtaining an input graph including a plurality of network components, where each of the plurality of network components include a plurality of nodes and a plurality of edges;
segmenting the input graph into a first partial input graph and a second partial input graph;
generating, using a first processor and a graph neural network (GNN), first network features based on the first partial input graph, wherein each of the first network features includes a connectivity relation between a first network component and an adjacent first network component among the plurality of network components in the input graph;
generating, using a second processor and the GNN, second network features based on the second partial input graph, wherein each of the second network features includes a connectivity relation between a second network component and an adjacent second network component among the plurality of network components in the input graph;
transmitting, among the first processor and the second processor, a network feature from the first partial input graph to an adjacent network feature in the second partial input graph to obtain an aggregated network feature; and
updating, using the second processor and a second layer of the GNN, the second network features based on the aggregated network feature.

12. The method of claim 11, wherein segmenting the input graph comprises:
segmenting the plurality of nodes of the input graph into a first node group and a second node group;
determining a first additional node group including a subset of nodes from the second node group and is adjacent to a node of the first node group among the plurality of nodes of the input graph;
determining a second additional node group including a subset of nodes from the first node group and is adjacent to a node of the second node group among the plurality of nodes of the input graph;
generating the first partial input graph based on the first node group and the first additional node group; and
generating the second partial input graph based on the second node group and the second additional node group.

13. An electronic device comprising:
a plurality of processors, wherein the plurality of processors is configured to:
obtain an input graph including a plurality of network components, where each of the plurality of network components include a plurality of nodes and a plurality of edges as a plurality of components,
segment the input graph into a plurality of partial input graphs, wherein the plurality of partial input graphs includes a first partial input graph and a second partial input graph,
generate network features based on the plurality of partial input graphs, wherein each of the network features includes a connectivity relation between a network component and an adjacent network component among the plurality of network components in the input graph,
transmit, among the plurality of processors, a network feature from the first partial input graph to an adjacent network feature in the second partial input graph to obtain an aggregated network feature, and
update the network feature of the plurality of network components based on the aggregated network feature.

14. The electronic device of claim 13, wherein the plurality of processors is further configured to:
compute, using a first processor of the plurality of processors, a first group of network features of the first partial input graph; and
compute, using a second processor of the plurality of processors, a second group of network features of the second partial input graph in a parallel state, wherein in the parallel state, the first processor and the second processor simultaneously computes the first group of network features and the second group of network features, respectively.

15. The electronic device of claim 13, wherein the plurality of processors is further configured to:
determine a target network component adjacent to the second partial input graph among network components of the first partial input graph based on the connectivity relation of the input graph, and
transmit a target network feature of the target network component to a first processor corresponding to the first partial input graph through a second processor, wherein the target network feature is generated based on the second processor corresponding to the second partial input graph.

16. The electronic device of claim 13, wherein the plurality of processors is further configured to:
segment the plurality of nodes of the input graph into a first node group and a second node group,
determine a first additional node group including a subset of nodes from the second node group and is adjacent to a node of the first node group among the plurality of nodes of the input graph,
determine a second additional node group including a subset of nodes from the first node group and is adjacent to a node of the second node group among the plurality of nodes of the input graph,
generate a first partial input graph based on the first node group and the first additional node group, and
generate a second partial input graph based on the second node group and the second additional node group.

17. The electronic device of claim 16, wherein the plurality of processors is further configured to:
determining a first number of nodes belonging to the first node group; and
determining a second number of nodes belonging to the second node group, wherein a difference between the first number and the second number is less than or equal to a threshold value.

18. The electronic device of claim 16, wherein the plurality of processors is further configured to:
obtain a first cumulative operation amount based on nodes belonging to the first node group and edges between the nodes; obtaining a second cumulative operation amount based on nodes belonging to the second node group and edges between the nodes; and
segment the plurality of network components based on the first cumulative operation amount and the second cumulative operation amount, where a difference between the first cumulative operation amount and the second cumulative operation amount is less than or equal to a threshold operation amount.

19. The electronic device of claim 13, wherein the plurality of processors is further configured to:
determine a number of first network components and a number of second network components based on a first operation amount of a first GNN and a second operation amount of a second GNN, respectively,
obtain the first partial input graph and the second partial input graph based on the number of first network components and the number of second network components, respectively,
compute network features of network components of the first partial input graph by applying one or more layers of the first GNN to the first partial input graph, and
compute network features of network components of the second partial input graph by applying one or more layers of the second GNN to the second partial input graph.

20. The electronic device of claim 13, wherein the plurality of processors is further configured to:
apply input data corresponding to the plurality of partial input graphs to an input layer of a graphic neural network (GNN),
perform forward propagation using the input data,
generate output data from an output layer of the GNN based on the forward propagation,
apply the output data to the output layer of the GNN,
perform backward propagation using the output data, and
generate additional output data from the input layer of the GNN based on the backward propagation.
